(19) United States
(12) Patent Application Publication
(10) Pub. No.: US 2016/0089252 A1
(43) Pub. Date: Mar. 31, 2016

(54) EXTRAVASCULAR PROTECTIVE STENT

(71) Applicant: Mingyuan CHEN, Guangdong (CN)

(72) Inventor: Mingyuan CHEN, Guangzhou City (CN)

(21) Appl. No.: 14/889,484

(22) PCT Filed: Apr. 29, 2014
(86) PCT No.: PCT/CN2014/076457

§ 371 Date: Nov. 6, 2015

(30) Foreign Application Priority Data
May 6, 2013 (CN) 201310162419.2
Mar. 19, 2014 (CN) 201410104084.3

(51) Int. Cl. A61F 2/82

(52) U.S. Cl. CPC A61F 2/82; A61F 2210/0071; A61F 2230/0023; A61F 2230/0069

(57) ABSTRACT

An extravascular protective stent includes a booster, a hollow protective tube and a hollow separator all sequentially arranged, wherein the side wall of the protective tube is provided with a protective notch parallel to the central axis of the protective tube and extending through two ends of the protective tube. During use of the separator and the protective tube when an internal carotid artery is exposed after a common carotid artery bifurcation is separated via a submandibular incision, the separator and the protective tube wrap the internal carotid artery in sequence, allowing the separating notch and the protective notch to face outward, and the closed wall thereof to face inward and directly face the surgery direction, such that a surgeon can thoroughly excise diseased tissue in surgery and avoid hemorrhage caused by accidentally injuring the internal carotid artery during the surgery, and improve the surgical efficacy.

### FIELD OF THE INVENTION

The present invention relates to the Field of medical appliances, and in particular, to a device for protecting blood vessels, and more particularly, to an extravascular protective stent which protects an internal carotid artery of a patient suffering partially recrudescent nasopharyngeal carcinoma after radiotherapy during a surgery treatment.

### BACKGROUND OF THE INVENTION

Reirradiation and salvage surgery are the only effective treatment ways for the management of locally recurrent nasopharyngeal carcinoma, but reirradiation not only has poor efficacy, but has severe radiation sequela; moreover, the five-year survival rate is 9%˜25% only. Salvage surgical resection which can directly remove radioresistant lesions, and avoid radiation-induced injury, is a preferable choice for locally recurrent nasopharyngeal carcinoma. Researches show that the efficacy of the surgical treatment is equal to and even higher than the reirradiation in case of same relapse restaging, and which benefits the patients with better quality of life. However, it should be noted that the parapharyngeal space which is frequently invaded by the nasopharyngeal carcinoma is lack of osseous protection, and an internal carotid artery passes through this space; therefore, a surgeon does not thoroughly excise the diseased tissue of a sufferer having a larger lesion range in order to prevent hemorrhage during the surgery caused by accidentally injuring the internal carotid artery, which causes surgery failure. Therefore, the internal carotid artery becomes a restrictive factor to nasopharyngeal carcinoma relief surgery, and is almost the unique hazard factor to the nasopharyngeal carcinoma relief surgery. An extracranial segment of the internal carotid artery is also called as cervical segment from a carotid artery bifurcation to a basis cranii, which is the longest among each segment of the internal carotid artery. The internal carotid artery firstly runs up at the posterior-exterior of an external carotid artery, then turns to the posterior-interior of the external carotid artery, and reaches the basis cranii along a lateral pharyngeal wall. It is characterized in that: (1) the extracranial segment has no ramification; (2) a starting segment has carotid sinus; and (3) the position is too deep to touch. At present, there is no surgical instrument capable of directly protecting the internal carotid artery in the market. An endovascular stent graft may be embedded via a method of interventional therapy for repairing injured internal carotid artery, but the endovascular stent graft is not only expensive, requires a special interventional physician and surgical instruments, and is complicated to operate during the surgery, but also cannot prevent rhexis of the internal carotid artery during the surgery, and does not belong to the scope of blood vessel protective instruments.

### SUMMARY OF THE INVENTION

The object of the present invention is to provide an extravascular protective stent, through which a surgical instrument in an internal carotid artery located in the parapharyngeal space can be distinguished and protected, so that the surgical risk is reduced to minimum, and the tumor can be excised to the maximum extent.

The technical problem to be solved by the present invention is as follows: an extravascular protective stent includes a booster, a hollow protective tube and a hollow separator all sequentially arranged, wherein the side wall of the protective tube is provided with a protective notch parallel to the central axis of the protective tube and extending through the two ends of the protective tube; the side wall of the separator is provided with a separating notch extending through the two ends of the separator; and the protective notch and the separating notch are symmetrically arranged in a straight line.

As a further improvement of the foregoing technical solution, the outer side wall of the protective tube opposite to the protective notch is provided with at least two scale floating points, and the scale floating points are arranged along the protective notch in a straight line, wherein one scale floating point is close to the booster and the other scale floating point is close to the separator.

As a further improvement of the foregoing technical solution, two scale floating points are arranged, and the scale floating points are arranged on the outer side wall of the protective tube equally.

As a further improvement of the foregoing technical solution, the outer wall of the separator is folded into a tapered lug boss along the direction far away from the protective tube.

As a further improvement of the foregoing technical solution, the cross section of the booster is of a broom-shaped triangle, and the narrow end of the booster is connected to the protective tube.

As a further improvement of the foregoing technical solution, both the junction of the booster and the protective tube and the junction of the protective tube and the separator are rounded off, and the exposed edges thereof are all blunt edges.

As a further improvement of the foregoing technical solution, the booster, the protective tube and the separator are all made of medical polyurethane.

The present invention has the advantageous effects that: during use of the separator and the protective tube when an internal carotid artery is exposed after a common carotid artery bifurcation is separated via submandibular the separator and the protective tube wrap the internal carotid artery in sequence, allowing the separating notch and the protective notch to face outward, and the closed wall thereof to face inward and directly face the surgery direction, such that a surgeon can thoroughly excise diseased tissue in surgery and avoid hemorrhage caused by accidentally injuring the internal carotid artery during the surgery, and improve the surgical efficacy.

### BRIEF DESCRIPTION OF THE DRAWINGS

In order to explain the technical solutions in the embodiments of the invention more clearly, the drawings used in the descriptions of the embodiments will be simply introduced hereinafter. Apparently, the drawings described are merely partial embodiments of the present invention, rather than all embodiments. Those skilled in the art may also obtain other design solutions and drawings according to these drawings without going through creative work.

FIG. 1 is a structure schematic view of a first viewing angle of the invention;

FIG. 2 is a structure schematic view of a second viewing angle of the invention; and

FIG. 3 is a top view of the invention.

### DETAILED DESCRIPTION OF THE EMBODIMENTS

The conceive, detailed structure and technical effects produced of the invention will be clearly and completely described hereinafter with reference to the embodiments and drawings, so as to sufficiently understand the object, characteristic and effect of the invention. Apparently, the embodiments described are merely partial embodiments of the present invention, rather than all embodiments; based on the embodiments of the invention, other embodiments obtained by those skilled in the art without going through creative work shall all belong to the protection scope of the invention. Moreover, all coupling/connecting relationship mentioned herein refers to forming a preferable coupling structure by means of adding or reducing coupling accessories according to the concrete implementation situations rather than direct coupling between members.

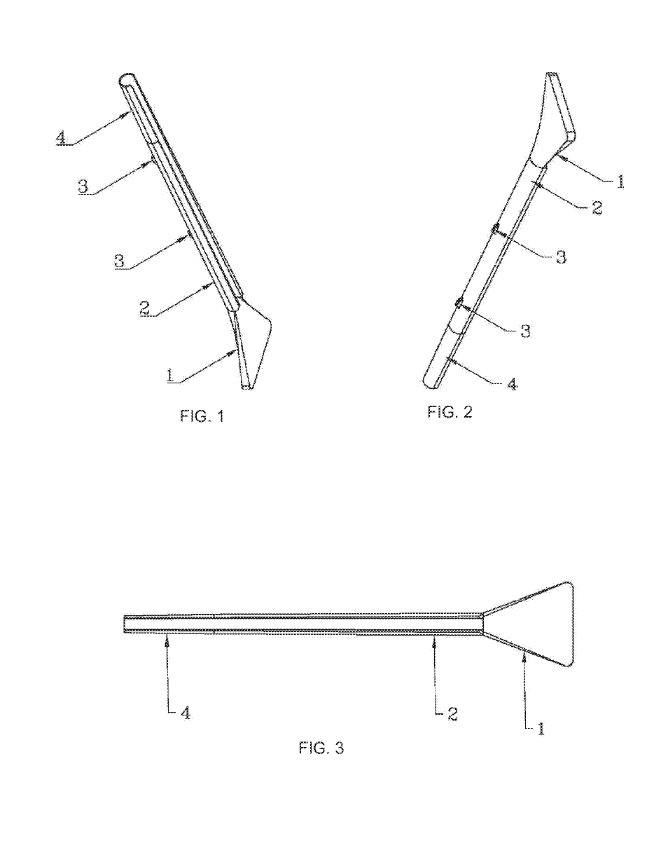

Referring to FIG. 1 to FIG. 3, an extravascular protective stent includes a booster 1, a hollow protective tube 2 and a hollow separator 4 all sequentially arranged, wherein the side wall of the protective tube 2 is provided with a protective notch parallel to the central axis of the protective tube 2 and extending through the two ends of the protective tube 2; the cross section of the protective tube 2 is “C”-shaped; the length of the protective tube 2 is about 9 cm; the side wall of the separator 4 is provided with a separating notch extending through the two ends of the separator 4; the cross section of the separator 4 is “C”-shaped; the length of the separator 4 is about 3 cm; the hardness of the separator 4 is slightly softer than that of the protective tube 2; and the protective notch and the separating notch are symmetrically arranged in a straight line.

During use, when an internal carotid artery is exposed after a common carotid artery bifurcation is separated via a submandibular incision, the separator 4 and the protective tube 2 wrap the internal carotid artery in sequence, allowing the separating notch and the protective notch to face outward, and the closed wall thereof to face inward and directly face the surgery direction, such that a surgeon can thoroughly excise diseased tissue in surgery and avoid hemorrhage caused by accidentally the internal carotid artery during the surgery, and improve the surgical efficacy.

As a further preferable embodiment, the outer side wall of the protective tube 2 opposite to the protective notch is provided with at least two scale floating points 3, and the scale floating points 3 are arranged along the protective notch in a straight line, wherein one scale floating point 3 is close to the booster 1 and one scale floating point 3 is close to the separator 4. An approach length may be estimated through the scale floating points 3, so as to estimate the approach part of the separator 4. The scale floating points 3 may also be served as force bearing points of a vessel clamp holder.

As a further preferable embodiment, two scale floating points 3 are arranged, and the scale floating points 3 are arranged on the outer side wall of the protective tube 2 equally.

As a further preferable embodiment, the outer wall of the separator 4 is folded into a tapered lug boss along the direction far away from the protective tube 2, so as to conveniently separate soft tissues surrounding the internal carotid artery and blood vessel.

As a further preferable embodiment, the cross section of the booster 1 is of a broom-shaped triangle, which complies with human mechanics; the length of the booster 1 is about 3 cm; and the narrow end of the booster 1 is connected to the protective tube 2.

As a further preferable embodiment, both the junction of the booster 1 and the protective tube 2 and the junction of the protective tube 2 and the separator 4 are rounded off, and the exposed edges thereof are all blunt edges.

As a further preferable embodiment, the booster 1, the protective tube 2 and the separator 4 are all made of medical polyurethane. The hardness of the booster 1, the protective tube 2 and the separator 4 are temperate, which will not accidentally injure the blood vessel due to over rigid and sharp factors, and will not fail to effectively separate the soft tissues surrounding the internal carotid artery and the blood vessel due to over soft factor. The booster 1, the protective tube 2 and the separator 4 have insulativity, and do not have exclusive reaction and sensitization.

When the internal carotid artery is exposed after the common carotid artery bifurcation is separated via a submandibular incision, the separator 4 and the protective tube 2 wrap the internal carotid artery in sequence, allowing the separating notch and the protective notch to face outward, and the closed wall thereof to face inward and directly face the surgery direction. During an initial operation stage, in order to prevent the protective tube 2 and the separator 4 from modification due to unequal force bearing, a pair of mosquito forceps may be employed to clinch the scale floating points 3 to propel the protective tube 2 and the separator 4. With the deepening of the protective tube 2 and the separator 4, the protective tube 2 and the separator 4 may be gradually propelled by the booster until the separator 4 reaches the basis cranii. After approaching to the basis cranii, in order to prevent the front segment of the stent and the internal carotid artery of the gap of the basis cranii from being accidentally injured, the protective tube 2 and the separator 4 may be continuously propelled, and the separator 4 is inserted into a canalis carotid's through an extracranial opening of the canalis caroticus to be seamlessly connected to the bone of the basis cranii, thus achieving the object of protecting the cervical segment of the internal carotid artery during the whole course.

The above is specific explanations to the preferred embodiments of the invention, but the invention is not limited to the embodiments. Those skilled in the an may also make various equivalent modifications or substitutions without departing from the spirit of the invention, and these equivalent modifications or substitutions shall all fall within the scope defined by the claims of the present application.

1. An extravascular protective stent, comprising a booster, a hollow protective tube and a hollow separator all sequentially arranged, wherein the side wall of the protective tube is provided with a protective notch parallel to the central axis of the protective tube and extending through two ends of the protective tube; the side wall of the separator is provided with a separating notch extending through two ends of the separator; and the protective notch and the separating notch are symmetrically arranged in a straight line.

2. The extravascular protective stent according to claim 1, wherein the outer side wall of the protective tube opposite to the protective notch is provided with at least two scale floating points, and the scale floating points are arranged along the protective notch in a straight line, wherein one scale floating point close to the booster and the other scale floating point is close to the separator.

3. The extravascular protective stent according to claim 2, wherein two scale floating points are arranged, and the scale floating points are arranged on the outer side wall of the protective tube equally.

4. The extravascular protective stent according to claim 1, wherein: the outer wall of the separator is folded into a tapered lug boss along the direction far away from the protective tube.

5. The extravascular protective stent according to claim 4, wherein the cross-section of the booster is of a broom-shaped triangle, and the narrow end of the booster is connected to the protective tube.

6. The extravascular protective stent according to claim 4, wherein both the junction of the booster and the protective tube and the junction of the protective tube and the separator are rounded off, and the exposed edges thereof are all blunt edges.

7. The extravascular protective stent according to claim 1, wherein the booster, the protective tube and the separator are all made of medical polyurethane.
